(19) United States
(12) Patent Application Publication
(10) Pub. No.: US 2021/0338642 A1
(43) Pub. Date: Nov. 4, 2021

(54) COMPOSITIONS AND METHODS FOR THE TREATMENT OR PREVENTION OF PULMONARY HYPERTENSION

(71) Applicant: Vivus, Inc., Campbell, CA (US)

(72) Inventors: Narinder S. BANAIT, San Carlos, CA (US); Leo GU, Saratoga, CA (US)

(21) Appl. No.: 17/373,006

(22) Filed: Jul. 12, 2021

Related U.S. Application Data
(63) Continuation of application No. 16/360,114, filed on Mar. 21, 2019, now abandoned.
Application No. 16/360,114 is a continuation of application No. 15/572,487, filed on Nov. 7, 2017, now abandoned.
(60) Provisional application No. 62/159,162, filed on May 8, 2015.

(51) Int. Cl. A61K 31/436; A61P 11/00

(52) U.S. Cl. CPC A61K 31/436; A61P 11/00

(57) ABSTRACT

The present invention provides compositions and methods for the treatment or prevention of pulmonary hypertension comprising administering an ascomycin, or a pharmaceutically acceptable salt, solvate, analog, or prodrug thereof to the patient with pulmonary hypertension.

### RELATED APPLICATIONS

This application is a continuation of U.S. application Ser. No. 16/360,114, filed Mar. 21, 2019, which is a continuation of U.S. application Ser. No. 15/572,487, filed Nov. 7, 2017, which is a national phase filing under 35 USC § 371 of International Application No. PCT/US2016/030737, entitled “COMPOSITIONS AND METHODS FOR THE TREATMENT OR PREVENTION OF PULMONARY HYPERTENSION”, filed May 4, 2016, which claims the priority benefit of U.S. Provisional Application No. 62/159,162, filed May 8, 2015, the contents of each of which are hereby incorporated by reference in their entireties.

### FIELD OF INVENTION

The present disclosure relates to methods for the treatment or prevention of pulmonary hypertension. In particular, the present disclosure relates to modulators of bone morphogenetic protein receptor type II (BMPR2), pharmaceutical formulations thereof and their use, alone or in combination with one or more additional agents, for treating and/or preventing various diseases, wherein an increase in the concentration of bone morphogenetic proteins (BMP) might be desirable.

### BACKGROUND

Pulmonary hypertension (PH) refers to a disease characterized by sustained elevations of pulmonary artery pressure. Generally, a patient having a mean pulmonary artery pressure equal to or greater than 25 mm Hg with a pulmonary capillary or left atrial pressure equal to or less than 15 mm Hg is characterized as having PH or as symptomatic of PH. These parameters may be measured in the subject at rest by right-heart catheterization.

The World Health Organization (WHO) has classified pulmonary hypertension into groups based on known causes. WHO group I includes patients with pulmonary arterial hypertension (PAH) including those patients with idiopathic PAH; familial PAH, and associated PAH, which is related to certain conditions including connective tissue diseases, congenital systemic-to-pulmonary-shunts, portal hypertension, HIV infection, drugs and toxins, glycogen storage disease, Gaucher's disease, hereditary hemorrhagic telangiectasia, hemoglobinopathies, myeloproliferative disorders, splenectomy, and others; PAH associated with significant venous or capillary involvement; and persistent pulmonary hypertension of the newborn. WHO group II includes patients with pulmonary venous hypertension. WHO group III includes patients with pulmonary hypertension associated with hypoxemia. WHO group IV includes patients with pulmonary hypertension due to chronic thrombotic disease, embolic disease or both. Finally, WHO group V includes patients with pulmonary hypertension due a variety of miscellaneous conditions.

The New York Heart Association (NYHA) further classifies pulmonary arterial hypertension into functional groups based on their exercise capacity and symptoms. NYHA functional class (FC) I includes patients with PAH without limitations of physical activity. FC II includes patients with PAH resulting in slight limitation of physical activity. FC III includes patients with PAH resulting in marked limitation in physical activity. FC IV includes patients with PAH that are unable to engage in physical activity without manifesting symptoms.

PAH is a serious, progressive and life-threatening disease of the pulmonary vasculature, characterized by vasoconstriction and an abnormal proliferation of smooth muscle cells in the walls of the pulmonary arteries. Severe constriction of the blood vessels in the lungs leads to very high pulmonary arterial pressures. These high pressures make it difficult for the heart to pump blood through the lungs to be oxygenated. Patients with PAH suffer from extreme shortness of breath as the heart struggles to pump against these high pressures. Patients with PAH typically develop significant increases in pulmonary vascular resistance (PVR) and sustained elevations in pulmonary artery pressure (PAP), which ultimately lead to right ventricular failure and death. Patients diagnosed with PAH have a poor prognosis and compromised quality of life, with a mean life expectancy of 2 to 5 years from the time of diagnosis if untreated.

PAH includes idiopathic pulmonary arterial hypertension; familial pulmonary arterial hypertension; pulmonary arterial hypertension in the setting of connective tissue diseases ((e.g., localized cutaneous systemic sclerosis (CREST syndrome), diffuse scleroderma, systemic lupus erythematosus, mixed connective tissue disease, and other less common diseases), portal hypertension, congenital left-to-right intracardiac shunts, and infection with the human immunodeficiency virus); and persistent pulmonary hypertension of the newborn.

Current therapies for pulmonary hypertension are unsatisfactory. These typically involve calcium channel antagonists, prostacyclins, prostacyclin receptor (IP receptor) agonist, endothelin receptor antagonists, phosphodiesterase-5 (PDE5) inhibitors, and long-term anticoagulant therapy. However, each treatment has limitations and side effects. Importantly, the current therapeutic approaches mainly provide symptomatic relief and some improvement of prognosis. In addition, the current therapies have either undesired side effects or inconvenient drug administration routes. Consequently, new therapies for the treatment or prevention of pulmonary hypertension are needed.

### SUMMARY

The present invention provides compositions and methods for the treatment of pulmonary hypertension, in particular pulmonary arterial hypertension.

In one aspect, the present invention describes a method of treating or preventing pulmonary hypertension in a patient in need thereof, the method comprising administering a therapeutically effective amount of a compound that increases BMPR2 signaling (BMPR2 activator) to the patient with pulmonary hypertension or a condition related thereto. The subject can be a mammal, such as a human. The BMPR2 activator can be ascomycin or a pharmaceutically acceptable salt, solvate, analog or prodrug thereof.

In another aspect, the present invention describes a method of treating or preventing pulmonary hypertension in a patient in need thereof, the method comprising administering a therapeutically effective amount of ascomycin or a pharmaceutically acceptable salt, solvate, analog, or prodrug thereof, to the patient with pulmonary arterial hypertension a condition related thereto.

In another aspect, the present invention describes a method of treating or preventing pulmonary hypertension in a patient in need thereof, the method comprising administering a therapeutically effective amount of a compound that increases BMPR2 signaling (BMPR2 activator) to the patient with pulmonary arterial hypertension. The BMPR2 activator is administered to improve exercise ability, delay clinical worsening, or combinations thereof.

These and other aspects of the present invention will become evident upon reference to the following detailed description

### BRIEF DESCRIPTION OF DRAWINGS

FIGS. 1A and 1B illustrate the changes in hemodynamic data for rats treated with SU5416 and hypoxia for 3 weeks (Sugen model), followed by treatment with vehicle (negative control), 0.05 mg/kg/day of tacrolimus (FK506), 0.05 mg/kg/day of ascomycin, or 0.15 mg/kg/day of ascomycin. The control group was not injected with SU5416. FIG. 1A shows the mean pulmonary arterial pressure upon sacrifice. FIG. 1B shows the right ventricle systolic pressure (RVSP) upon sacrifice.

FIGS. 2A and 2B illustrate the changes in hemodynamic data for rats treated with SU5416 and hypoxia for 3 weeks (Sugen model), followed by 3 weeks of normoxia and then treatment with vehicle (negative control), 0.05 mg/kg/day of tacrolimus (FK506), or 0.3 mg/kg/day, 1.0 mg/kg/day, or 3.0 mg/kg/day of ascomycin (FK520). The control group was not injected with SU5416. FIG. 2A shows the mean pulmonary arterial pressure upon sacrifice. FIG. 2B shows the right ventricle systolic pressure (RVSP) upon sacrifice.

FIGS. 3A and 3B illustrate the morphological data. FIG. 3A shows micrographs from the lungs of the negative control animals. FIG. 3B shows micrographs from rats treated with 1 mg/kg/day of ascomycin.

### DETAILED DESCRIPTION

### I. Definitions

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Definition of standard chemistry terms may be found in reference works, including Carey and Sundberg (2004) “Advanced Organic Chemistry 4^{rd} Ed.” Vols. A and B, Springer, New York. The practice of the present invention will employ, unless otherwise indicated, conventional methods of mass spectroscopy, protein chemistry, biochemistry, and pharmacology, within the skill of the art.

The term “modulator” means a molecule that interacts with a target. The interactions include, but are not limited to, agonist, antagonist, and the like, as defined herein.

The term “agonist” means a molecule such as a compound, a drug, an enzyme activator or a hormone that enhances the activity of another molecule or the activity of the target receptor.

The term “antagonist” means a molecule such as a compound, a drug, an enzyme inhibitor, or a hormone, that diminishes or prevents the action of another molecule or the activity of the target receptor.

The terms “effective amount” or “pharmaceutically effective amount” refer to a sufficient amount of the agent to provide the desired biological result without an unacceptable toxic effect. That result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in a disease. An appropriate “effective” amount in any individual case may be determined by one of ordinary skill in the art using routine experimentation.

As used herein, the terms “treat” or “treatment” are used interchangeably and are meant to ameliorating the disease or disorder (i.e., arresting or reducing the development of the disease or at least one of the clinical symptoms thereof). In one embodiment “treating” or “treatment” refers to ameliorating at least one symptom of the disease. In another embodiment, “treating” or “treatment” refers to inhibiting the disease or disorder, either physically (e.g., stabilization of a discernible symptom), physiologically, (e.g., stabilization of a physical parameter), or both.

By “pharmaceutically acceptable” or “pharmacologically acceptable” is meant a material which is not biologically or otherwise undesirable, i.e., the material may be administered to an individual without causing any undesirable biological effects or interacting in a deleterious manner with any of the components of the composition in which it is contained.

As used herein, the term “mammal subject” encompasses any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like.

The term “pharmaceutically acceptable salt” of a compound means a salt that is pharmaceutically acceptable and that possesses the desired pharmacological activity of the parent compound. Such salts, for example, include:

(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like;

(2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate, sodium hydroxide, and the like. It should be understood that a reference to a pharmaceutically acceptable salt includes the solvent addition forms or crystal forms thereof, particularly solvates or polymorphs. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

The term “optional” or “optionally” means that the subsequently described event or circumstance may or may not occur, and that the description includes instances where the event or circumstance occurs and instances where it does not.

All publications, patents and patent applications cited herein, whether supra or infra, are hereby incorporated by reference in their entirety.

### II. Description of the Invention

The compositions and methods of the present invention increase signaling of the BMPR2 pathway. Thus, the present invention provides compositions and methods for the prevention or treatment of a BMPR2 pathway mediated condition or disease. The BMPR2 pathway is an important pathway, the expression of which is reduced in patients with pulmonary arterial hypertension (PAH). Therefore, increasing BMPR2 signaling in patients with PAH can prevent or reverse disease.

In particular, the present invention provides for the use of a compound for the treatment of PAH selected from: idiopathic PAH; familial PAH; PAH associated with a collagen vascular disease selected from: scleroderma, CREST syndrome, systemic lupus erythematosus (SLE), rheumatoid arthritis, Takayasu's arteritis, polymyositis, and dermatomyositis; PAH associated with a congenital heart disease selected from: atrial septic defect (ASD), ventricular septic defect (VSD) and patent ductus arteriosus in an individual; PAH associated with portal hypertension; PAH associated with HIV infection; PAH associated with ingestion of a drug or toxin; PAH associated with hereditary hemorrhagic telangiectasia; PAH associated with splenectomy; PAH associated with significant venous or capillary involvement; PAH associated with pulmonary veno-occlusive disease (PVOD); and PAH associated with pulmonary capillary hemangiomatosis (PCH).

In one aspect, compositions and methods of treating or preventing pulmonary hypertension are described comprising administering a therapeutically effective amount of ascomycin or a pharmaceutically acceptable salt, solvate, analog, or prodrug thereof. The IUPAC name of ascomycin is (3S,4R,5S,8R,9E,12S,14S,15R,16S,18R,19R,26aS)-8-ethyl-5,19-dihydroxy-3-{(1E)-1-[(1R,3R,4R)-4-hydroxy-3-methoxycyclohexyl]prop-1-en-2-yl}-14,16-dimethoxy-4,10,12,18-tetramethyl-5,6,8,11,12,13,14,15,16,17,18,19,24,25,26,26a-hexadecahydro-3H-15,19-epoxypyrido[2,1-c][1,4]oxazacyclotricosine-1,7,20,21(4H,23H)-tetrone. Ascomycin has the formula C₄₃H₆₉NO₁₂, and the structure is shown below:

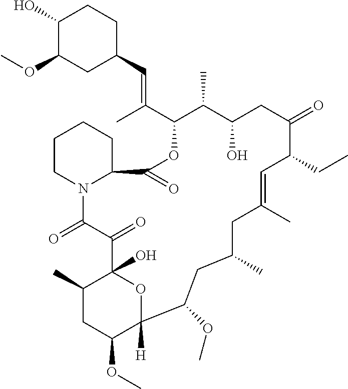 [SMILES: CC[C@@H]1/C=C(\C)C[C@H](C)C[C@H](OC)[C@H]2O[C@@](O)(C(=O)C(=O)N3CCCC[C@H]3C(=O)O[C@H](/C(C)=C/[C@@H]3CC[C@@H](O)[C@H](OC)C3)[C@H](C)[C@@H](O)CC1=O)[C@H](C)C[C@@H]2OC]

Ascomycin is a substantial component of a large family of macrocyclic fermentation products. This family, originally isolated from cultures of Streptomyces hygroscopicus var. ascomyceticus (T. Arai, et al., J. Antibiotics (Tokyo) 15 (Ser. A), 231-232 (1962)), is produced as a group of secondary fungal metabolites. Ascomycin was initially investigated for its antifungal activities but it is also an immunosuppressant, acting primarily through T-lymphocytes via inhibition of the phosphatase calcineurin. Ascomycin reduces the production of a range of cytokines, inhibiting the activation of various cell types, including those involved in cell-mediated immunity. Due to these properties, ascomycin was considered for the development of therapeutics in the transplantation field to help prevent rejection after an organ transplant, and for the treatment of autoimmune diseases and skin diseases.

Ascomycin, also called Immunomycin, FR-900520, FK520, has an IC50 Value: 0.55 nM. Ascomycin had a 3-fold lower immunosuppressive potency in a popliteal lymph node hyperplasia assay compared with tacrolimus. In 14-day studies, nephrotoxicity was not induced by continuous i.p. infusion of ascomycin at 10 mg/kg/day or daily oral administration (up to 50 mg/kg/day) in rats on a normal diet, nor by continuous i.v. infusion (up to 6 mg/kg/day) in rats on a low salt diet to enhance susceptibility Mollison et al. Toxicology (1998) 125 (2-3): 169-181.

Ascomycin was shown to activate BMP signaling using a C2C12 mouse myoblastoma reporter cell line stably transfected with a reporter plasmid expressing a BMP response element (BRE) from the Id1 promoter (a main downstream target of BMP signaling) fused to the luciferase-gene (BRE-luc) using BMP4 as a positive control. The activation of the BMP pathway was measured by luminescence. Ascomycin increased ID1 activation by 104% without exogenous BMP4 and 238% with exogenous BMP4, where the values of percentage of activation are defined as luminescence relative to the EC-20 dose of BMP4. Thus, ascomycin activates BMPR2 signaling, and can be used for the prevention or treatment of diseases associated with decreased BMPR2 signaling, such as pulmonary arterial hypertension (PAH).

In another aspect, compositions and methods of treating or preventing pulmonary hypertension comprise administering a therapeutically effective amount of an ascomycin analog, such as pimecrolimus (33-epi-chloro-33-desoxy-ascomycin), ABT-281, SDZ 281-240, FK506 (tacrolimus), FK523 (desmethyl acomycin), FK525 (prolytacrolimus), 32-O-(1-hydroxyethylindol-5-yl)-ascomycin, 18-OH ascomycin, 18-ene-20-oxa-ascomycin and its 13-desmethoxy-13-methyl analogue, 13-dM(Me)-ascomycin, 13-dM(Me)-18-OH-ascomycin, 13-dM(Me)-18-ene-20-oxa-ascomycin, 31-desmethoxy-ascomycin, 31-desmethoxy-31-cis-hydroxy-32-trans-hydroxy-ascomycin, 31-desmethoxy-31-cis-hydroxy-32-cis-hydroxy-ascomycin, 31-desmethoxy-31-trans-hydroxy-32-trans-hydroxy-ascomycin, 31-O-desmethyl-32-dehydroxy-ascomycin, 31-O-desmethyl-ascomycin, 31-desmethoxy-31-methyl-ascomycin, 31-O-desmethyl-32-dehydroxy-32-methyl-ascomycin, 31-O-desmethyl-32-dehydroxy-32-fluoro-ascomycin, 31-desmethoxy-31-fluoro-ascomycin, 31-O-desmethyl-32-dehydroxy-32-chloro-ascomycin, 31-desmethoxy-31-chloro-ascomycin, 31-O-desmethyl-32-dehydroxy-32-tert-butyl-ascomycin, 29-de(3-methoxy-4-hydroxy-cyclohexyl)-29-(hydroxy-cycloheptyl)-ascomycin, 29-de(3-methoxy-4-hydroxy-cyclohexyl)-29-(hydroxy-norbornyl)-ascomycin, 9-deoxo-31-desmethoxy-ascomycin, 9-deoxo-31-desmethoxy-31-cis-hydroxy-32-trans-hydroxy-ascomycin, 9-deoxo-31-desmethoxy-31-cis-hydroxy-32-cis-hydroxy-ascomycin, 9-deoxo-31-desmethoxy-31-trans-hydroxy-32-trans-hydroxy-ascomycin, 9-deoxo-31-O-desmethyl-32-dehydroxy-ascomycin, 9-deoxo-31-O-desmethyl-ascomycin, 9-deoxo-31-desmethoxy-31-methyl-ascomycin, 9-deoxo-31-O-desmethyl-32-dehydroxy-32-methyl-ascomycin, 9-deoxo-31-O-desmethyl-32-dehydroxy-32-fluoro-ascomycin, 9-deoxo-31-desmethoxy-31-fluoro-ascomycin, 9-deoxo-31-O-desmethyl-32-dehydroxy-32-chloro-ascomycin, 9-deoxo-31-desmethoxy-31-chloro-ascomycin, 9-deoxo-31-O-desmethyl-32-dehydroxy-32-tert-butyl-ascomycin, 9-deoxo-29-de(3-methoxy-4-hydroxy-cyclohexyl)-29-(hydroxy-cycloheptyl-ascomycin, 9-deoxo-29-de(3-methoxy-4-hydroxy-cyclohexyl)-29-(hydroxy-norbornyl-ascomycin, 30-desmethoxy-prolyl-ascomycin, 30-desmethoxy-30-cis-hydroxy-31-trans-hydroxy-prolyl-ascomycin, 30-desmethoxy-30-cis-hydroxy-31-cis-hydroxy-prolyl-ascomycin, 30-desmethoxy-30-trans-hydroxy-31-trans-hydroxy-prolyl-ascomycin, 30-O-desmethyl-31-dehydroxy-prolyl-ascomycin, 30-O-desmethyl-prolyl-ascomycin, 30-desmethoxy-30-methyl-prolyl-ascomycin, 30-O-desmethyl-31-dehydroxy-31-methyl-prolyl-ascomycin, 30-O-desmethyl-31-dehydroxy-31-fluoro-prolyl-ascomycin, 30-desmethoxy-30-fluoro-prolyl-ascomycin, 30-O-desmethyl-31-dehydroxy-31-chloro-prolyl-ascomycin, 30-desmethoxy-30-chloro-prolyl-ascomycin, 30-desmethyl-31-dehydroxy-31-tert-butyl-prolyl-ascomycin, 28-de(3-methoxy-4-hydroxy-cyclohexyl)-28-(hydroxy-cycloheptyl-ascomycin, 28-de(3-methoxy-4-hydroxy-cyclohexyl)-28-(hydroxy-norbornyl-ascomycin, 8-deoxo-30-desmethoxy-31-hydroxy-prolyl-ascomycin, 8-deoxo-30-desmethoxy-30-cis-hydroxy-31-trans-hydroxy-prolyl-ascomycin, 8-deoxo-30-desmethoxy-30-cis-hydroxy-31-cis-hydroxy-prolyl-ascomycin, 8-deoxo-30-desmethoxy-30-trans-hydroxy-31-trans-hydroxy-prolyl-ascomycin, 8-deoxo-30-O-desmethyl-31-dehydroxy-prolyl-ascomycin, 8-deoxo-30-O-desmethyl-prolyl-ascomycin, 8-deoxo-30-desmethoxy-30-methyl-prolyl-ascomycin, 8-deoxo-30-O-desmethyl-31-dehydroxy-31-methyl-prolyl-ascomycin, 8-deoxo-30-O-desmethyl-31-dehydroxy-31-fluoro-prolyl-ascomycin, 8-deoxo-30-desmethoxy-30-fluoro-prolyl-ascomycin, 8-deoxo-30-O-desmethyl-31-dehydroxy-31-chloro-prolyl-ascomycin, 8-deoxo-30-desmethoxy-30-chloro-prolyl-ascomycin, 8-deoxo-30-O-desmethyl-31-dehydroxy-31-tert-butyl-prolyl-ascomycin, 8-deoxo-28-de(3-methoxy-4-hydroxy-cyclohexyl)-28-(hydroxy-cycloheptyl)-pr-olyl-ascomycin, 8-deoxo-28-de(3-methoxy-4-hydroxy-cyclohexyl)-28-(hydroxy-norbornyl)-prolyl-ascomycin, 30-desmethoxy-3-hydroxy-prolyl-ascomycin, 30-desmethoxy-30-cis-hydroxy-31-trans-hydroxy-3-hydroxy-prolyl-ascomycin, 30-desmethoxy-30-cis-hydroxy-31-cis-hydroxy-3-hydroxy-prolyl-ascomycin, 30-desmethoxy-30-trans-hyd-ascomycin roxy-31-trans-hydroxy-3-hydroxy-prolyl-ascomycin, 30-O-desmethyl-31-dehydroxy-3-hydroxy-prolyl-ascomycin, 30-O-desmethyl-3-hydroxy-prolyl-ascomycin, 30-desmethoxy-30-methyl-3-hydroxy-prolyl-ascomycin, 30-O-desmethyl-31-dehydroxy-31-methyl-3-hydroxy-prolyl-ascomycin, 30-O-desmethyl-31-dehydroxy-31-fluoro-3-hydroxy-prolyl-ascomycin, 30-desmethoxy-30-fluoro-3-hydroxy-prolyl-ascomycin, 31-desmethoxy-trans-3-bicyclo[3.1.0.]ascomycin, 31-desmethoxy-31-cis-hydroxy-32-trans-hydroxy-trans-3-bicyclo[3.1.0.]ascomycin, 31-desmethoxy-31-cis-hydroxy-32-cis-hydroxy-trans-3-bicyclo[3.1.0.]ascomycin, 31-desmethoxy-31-trans-hydroxy-32-trans-hydroxy-trans-3-bicyclo[3.1.0.]ascomycin, 31-desmethoxy-31-cis-hydroxy-32-trans-hydroxy-ascomycin, 31-desmethoxy-31-cis-hydroxy-32-cis-hydroxy-ascomycin, 31-desmethoxy-31-trans-hydroxy-32-trans-hydroxy-ascomycin, 31-desmethoxy-31-methyl-ascomycin, 31-desmethoxy-31-fluoro-ascomycin, 31-desmethoxy-31-chloro-ascomycin, 31-O-desmethyl-32-dehydroxy-32-tert-butyl-ascomycin, 29-de(3-methoxy-4-hydroxy-cyclohexyl)-29-(hydroxy-cycloheptyl)-ascomycin, 29-de(3-methoxy-4-hydroxy-cyclohexyl)-29-(hydroxy-norbornyl)-ascomycin, 9-deoxo-31-desmethoxy-31-cis-hydroxy-32-trans-hydroxy-ascomycin, 9-deoxo-31-desmethoxy-31-cis-hydroxy-32-cis-hydroxy-ascomycin, 9-deoxo-31-desmethoxy-31-trans-hydroxy-32-trans-hydroxy-ascomycin, 9-deoxo-31-desmethoxy-31-methyl-ascomycin, and the like. Preferred ascomycin analogs are pimecrolimus (33-epi-chloro-33-desoxy-ascomycin), FK523 (desmethyl acomycin), FK525 (prolytacrolimus), and FK 506 (tacrolimus).

The present invention also provides prodrugs of ascomycin and its analoges wherein the prodrug converts in vivo to ascomycin or its analoges. A prodrug is an active or inactive compound that is modified chemically through in vivo physiological action, such as hydrolysis, metabolism and the like, into a compound of this invention following administration of the prodrug to a subject. The suitability and techniques involved in making and using pro-drugs are well known by those skilled in the art. Prodrugs can be conceptually divided into two non-exclusive categories, bioprecursor prodrugs and carrier prodrugs (The Practice of Medicinal Chemistry, Ch. 31-32 (Ed. Wermuth, Academic Press, San Diego, Calif., 2001)). Generally, bioprecursor prodrugs are compounds which are inactive or have low activity compared to the corresponding active drug compound that contain one or more protective groups and are converted to an active form by metabolism or solvolysis. Both the active drug form and any released metabolic products should have acceptably low toxicity. Carrier prodrugs are drug compounds that contain a transport moiety, e.g., that improve uptake and/or localized delivery to a site(s) of action.

Exemplary prodrugs are, for example, esters of free carboxylic acids and S-acyl derivatives of thiols and O-acyl derivatives of alcohols or phenols, wherein acyl has a meaning as defined herein. Suitable prodrugs are often pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters. In addition, amines have been masked as arylcarbonyloxymethyl substituted derivatives which are cleaved by esterases in vivo releasing the free drug and formaldehyde (Bundgaard, J. Med. Chem. 2503 (1989)). Moreover, drugs containing an acidic NH group, such as imidazole, imide, indole and the like, have been masked with N-acyloxymethyl groups (Bundgaard, Design of Prodrugs, Elsevier (1985)). Hydroxy groups have been masked as esters and ethers.

Any compound given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds as defined above include isotopes of hydrogen, carbon, nitrogen, oxygen, fluorine, phosphorous, chlorine, and iodine such as ²H, ³H, ¹¹C, ¹³C, ¹⁴C, ¹⁵N, ¹⁵O, ¹⁷O, ¹⁸O, ¹⁸F, ³¹P, ³²P, ³⁵S, ³⁶Cl, and ¹²⁵I. Isotopically labeled compounds of this invention and prodrugs thereof can generally be prepared by carrying out the synthetic procedures by substituting a readily available isotopically labeled reagent for a non-isotopically labeled reagent. Isotopically-labeled compounds can generally be prepared by conventional techniques known to those skilled in the art using an appropriate isotopically-labeled reagent in place of the non-labeled reagent previously employed.

The compounds disclosed above, in free form, may be converted into salt form, and vice versa, in a conventional manner understood by those skilled in the art. The compounds in free or salt form can be obtained in the form of hydrates or solvates containing a solvent used for crystallization. The compounds can be recovered from reaction mixtures and purified in a conventional manner. Pharmaceutically acceptable solvates in accordance with the invention include those wherein the solvent of crystallization may be isotopically substituted, e.g. D₂O, d₆-acetone, d₆-DMSO.

Ascomycin, or a pharmaceutically acceptable salt, solvate, analog, or prodrug thereof, that increases BMPR2 signaling can be administered to a patient for the treatment or prevention of pulmonary hypertension, in particular PAH. Treatment or prevention of PAH as used herein encompasses one or more of the following:

(a) adjustment of one or more hemodynamic parameters towards a more normal level, for example lowering mean PAP or PVR, or raising PCWP or LVEDP, versus baseline;

(b) improvement of pulmonary function versus baseline, for example increasing exercise capacity, illustratively as measured in a test of 6-minute walking distance (6MWD), or lowering Borg dyspnea index (BDI);

(c) improvement of one or more quality of life parameters versus baseline, for example an increase in score on at least one of the SF-36™ health survey functional scales;

(d) general improvement versus baseline in the severity of the condition, for example by movement to a lower WHO functional class;

(e) improvement of clinical outcome following a period of treatment, versus expectation in absence of treatment (e.g., in a clinical trial setting, as measured by comparison with placebo), including improved prognosis, extending time to or lowering probability of clinical worsening, extending quality of life (e.g., delaying progression to a higher WHO functional class or slowing decline in one or more quality of life parameters such as SF-36™ health survey parameters), and/or increasing longevity; and/or

(f) adjustment towards a more normal level of one or more molecular markers that can be predictive of clinical outcome, such as plasma concentrations of bone morphogenetic protein (BMP), cardiac troponin T (cTnT), NT-proBNP, B-type natriuretic peptide (BNP), and the like.

Ascomycin, or a pharmaceutically acceptable salt, solvate, analog, or prodrug thereof can be administered in a therapeutically effective amount sufficient to provide any one or more of the effects mentioned above. Preferably the amount administered does not exceed an amount causing an unacceptable degree of adverse side effects. The therapeutically effective amount can vary depending on the compound, the particular pulmonary hypertension condition to be treated, the severity of the condition, body weight and other parameters of the individual subject, and can be readily established without undue experimentation by the physician or clinician based on the disclosure herein. Typically, a therapeutically effective amount will be found in the range of about 0.1 to about 100 mg/day, for example about 0.5 to about 75 mg/day, about 1 to about 10 mg/day, or about 0.5, about 1, about 1.5, about 2, about 2.5, about 3, about 3.5, about 4, about 4.5, about 5, about 6, about 7, about 8, about 9 or about 10 mg/day. The therapeutically effective amount can be administered each day, for example in individual doses administered once, twice, or three or more times a day. The therapeutically effective amount can be administered once each day, once every other day, or once every third day.

For example, if the compound to increase BMPR2 signaling is ascomycin or a pharmaceutically acceptable solvate, salt, analog, or prodrug thereof, it can be administered at a dose and regimen that provides ascomycin serum concentration or whole blood concentration of about 0.05 ng/mL to about 500 ng/mL, such as about 10 ng/mL, about 20 ng/mL, about 30 ng/mL, about 40 ng/mL, about 50 ng/mL, about 200 ng/mL; about 0.1 ng/mL to about 0.5 ng/mL, about 0.15 ng/mL to about 0.3 ng/mL or about 0.1-0.2 ng/mL. This is in part because ascomycin is metabolized by the cytochrome P450 system, so the exact dosing may vary between patients. Ascomycin or a pharmaceutically acceptable solvate, salt, analog, or prodrug thereof can be administered once, twice, or three or more times a day. In one aspect of the invention, the goal is to reach a blood level of about 0.2 ng/mL to about 100 ng/mL. In this case, an initial dose of 0.001 mg/kg to 0.5 mg/kg (e.g., 0.002 mg/kg/day to 1 mg/kg/day) may be sufficient, and the does can be up-titrated according to the measured ascomycin blood level. In particular cases, ascomycin can reach a blood concentration of <1.0 ng/mL, 1.5-2.5 ng/mL, 3-5 ng/mL, 10-12 ng/mL, 30-40 ng/mL, 50-60 ng/mL, 20 ng/mL, 30 ng/mL, 50 ng/mL, 100 ng/mL, 200 ng/mL, or 300 ng/mL.

The active agent to increase BMPR2 signaling can be administered in monotherapy. Alternatively, the compound to increase BMPR2 signaling can be administered in combination therapy with one or more other active agent effective for the treatment of the pulmonary hypertension condition or a condition related thereto. When a second or more active agent is administered concomitantly, one of skill in the art can readily identify a suitable dose for any particular second active agent from publicly available information in printed or electronic form, for example on the internet. Illustratively and without limitation, the active agent to increase BMPR2 signaling can be administered with a second active agent comprising at least one drug selected from the group consisting of prostanoids, phosphodiesterase inhibitors, especially phosphodiesterase-5 (PDE5) inhibitors, endothelin receptor antagonists (ERAs), prostacyclin receptor (IP receptor) agonist, soluble guanylate cyclase stimulator, calcium channel blockers, ASK-1 inhibitor, an inhibitor of proliferative signaling, an inhibitor of inflammatory signaling, diuretics, anticoagulants, nitric oxide, oxygen and combinations thereof.

In one aspect, ascomycin, or a pharmaceutically acceptable salt, solvate, analog, or prodrug thereof can be administered alone or in combination with other active compounds. Thus, compounds that increase the signaling of the BMPR2 pathway can further be combined with other compounds that increase vasodilation such as compounds that target endothelin (TRACLEER®, OPSUMIT®, and LETAIRIS®), nitric oxide/PDE-5 (REVATIO®, ADCIRCA®, avanafil, lodenafil, mirodenafil, udenafil, and zaprinast), prostacyclin (REMODULIN®, TYVASO®, and FLOLAN®), prostacyclin receptor agonists (selexipag, and APD811), soluble guanylate cyclase (RIOCIGUAT®), and the like. Thus, the combined compounds can become more effective agents for the treatment of PAH, and may provide additive or synergistic results from the combined use of the compounds that increase the signaling of the BMPR2 pathway with compounds that target other pathways.

Examples of drugs useful in combination therapy are classified and presented in several lists below. Some drugs are active at more than one target; accordingly, certain drugs may appear in more than one list. Use of any listed drug in a combination is contemplated herein, independently of its mode of action.

A suitable prostanoid can be illustratively selected from the following list: beraprost, cicaprost, epoprostenol, iloprost, NS-304, PGE₁ prostacyclin, and treprostinil.

A suitable PDE5 inhibitor can illustratively be selected from the following list: sildenafil, tadalafil, vardenafil, avanafil, lodenafil, mirodenafil, udenafil, and zaprinast.

A suitable ERA other than ambrisentan can illustratively be selected from the following list: atrasentan, ambrisentan, BMS 193884, bosentan, CI-1020, darusentan, S-0139 SB-209670, sitaxsentan, TA-0201, tarasentan, TBC-3711, VML-588, and ZD-1611.

A suitable ASK-1 inhibitor can illustratively be selected from GSK-4997 or GSK 444217.

A suitable inhibitor of proliferative signaling can illustratively be selected from imatinib or nilotinib.

A suitable inhibitor of inflammatory signaling can illustratively be selected from ubenimex or bardoxolone methyl.

A suitable calcium channel blocker can illustratively be selected from the following list: Aryklalkylamines: bepridil, clentiazem, diltiazem, fendiline, gallopamil, mibefradil, prenylamine, semotiadil, terodiline, and verapamil; Dihydropyridine, derivatives: amlodipine, aranidipine, barnidipine, benidipine, cilnidipine, efonidipine, elgodipine, felodipine, isradipine, lacidipine, lercanidipine, manidipine, nicardipine, nifedipine, nilvadipine, nimodipine, nisoldipine, nitrendipine, and NZ 105; Piperazine derivatives: cinnarizine, dotarizine, flunarizine, lidoflazine, and lomerizine; and Unclassified: bencyclane, etafenone, fantofarone, monatepil, perhexiline. Particularly suitable calcium channel blockers include amlodipine, diltiazem, felodipine, isradipine, nicardipine, nifedipine, nisoldipine, verapamil and combinations thereof.

A suitable diuretic can illustratively be selected from the following list: Organomercurials: chlormerodrin, chlorothiazide, chlorthalidone, meralluride, mercaptomerin, sodium mercumatilin, sodium mercurous, and chloride mersalyl; Purines: pamabrom, protheobromine, and theobromine; Steroids: canrenone, oleandrin, and spironolactone; Sulfonamide derivatives: acetazolamide, ambuside, azosemide, bumetanide, butazolamide, chloraminophenamide, clofenamide, clopamide, clorexolone, disulfamide, ethoxzolamide, furosemide, mefruside, methazolamide, piretanide, torsemide, tripamide, and xipamide; Thiazides and analogs: althiazide, bendroflumethiazide, benzthiazide, benzylhydrochlorothiazide, buthiazide, chlorthalidone, cyclopenthiazide, cyclothiazide, ethiazide, fenquizone, hydrochlorothiazide, hydroflumethiazide, indapamide, methyclothiazide, metolazone, paraflutizide, polythiazide, quinethazone, teclothiazide, and trichlormethiazide; Uracils: aminometradine; Unclassified: amiloride, Biogen BG 9719, chlorazanil, ethacrynic acid, etozolin, isosorbide, Kiowa Hakko KW 3902, mannitol, muzolimine, perhexiline, Sanofi-Aventis SR 121463, ticrynafen, triamterene, and urea. In some embodiments, the diuretic if present comprises a thiazide or loop diuretic. Thiazide diuretics are generally not preferred where the patient has a complicating condition such as diabetes or chronic kidney disease, and in such situations a loop diuretic can be a better choice. Particularly suitable thiazide diuretics include chlorothiazide, chlorthalidone, hydrochlorothiazide, indapamide, metolazone, polythiazide and combinations thereof. Particularly suitable loop diuretics include bumetanide, furosemide, torsemide and combinations thereof.

A suitable anticoagulant can illustratively be selected from the following list: acenocoumarol, ancrod, anisindione, bromindione, clorindione, coumetarol, cyclocumarol, dextran sulfate, sodium dicumarol, diphenadione, ethyl biscoumacetate, ethylidene dicoumarol, fluindione, heparin, hirudin, lyapolate, sodium pentosan, polysulfate phenindione, phenprocoumon, phosvitin, picotamide, tioclomarol, and warfarin.

Where the pulmonary hypertension condition is associated with an underlying disease (for example CTD, HIV infection, COPD or ILD), the active agent to increase BMPR2 signaling can optionally be administered in combination therapy with one or more drugs targeting the underlying condition.

When the active agent to increase BMPR2 signaling is used in combination therapy with one or more drugs, the active agent and at least one drug can be administered at different times or at about the same time (at exactly the same time or directly one after the other in any order). The active agent and the second active drug can be formulated in one dosage form as a fixed-dose combination for administration at the same time, or in two or more separate dosage forms for administration at the same or different times.

Separate dosage forms can optionally be co-packaged, for example in a single container or in a plurality of containers within a single outer package, or co-presented in separate packaging (“common presentation”). As an example of co-packaging or common presentation, a kit is contemplated comprising, in separate containers, active agent to increase BMPR2 signaling and at least one drug useful in combination with the active agent. In another example, the active agent and the at least one drug useful in combination therapy with the active agent are separately packaged and available for sale independently of one another, but are co-marketed or co-promoted for use according to the invention. The separate dosage forms can also be presented to a patient separately and independently, for use according to the invention.

### IV. Formulations

The compounds described above are preferably used to prepare a medicament, such as by formulation into pharmaceutical compositions for administration to a subject using techniques generally known in the art. A summary of such pharmaceutical compositions may be found, for example, in Remington's Pharmaceutical Sciences, Mack Publishing Co., Easton, Pa. The compounds of the invention can be used singly or as components of mixtures. Preferred forms of the compounds are those for systemic administration as well as those for topical or transdermal administration. Formulations designed for timed release are also with the scope of the invention. Formulation in unit dosage form is also preferred for the practice of the invention.

In unit dosage form, the formulation is divided into unit doses containing appropriate quantities of one or more compound. The unit dosage may be in the form of a package containing discrete quantities of the formulation. Non-limiting examples are packeted tablets or capsules, and powders in vials or ampoules.

The compounds of the invention may be labeled isotopically (e.g. with a radioisotope) or by another other means, including, but not limited to, the use of chromophores or fluorescent moieties, bioluminescent labels, or chemiluminescent labels. The compositions may be in conventional forms, either as liquid solutions or suspensions, solid forms suitable for solution or suspension in a liquid prior to use, or as emulsions. Suitable excipients or carriers are, for example, water, saline, dextrose, glycerol, alcohols, aloe vera gel, allantoin, glycerin, vitamin A and E oils, mineral oil, propylene glycol, PPG-2 myristyl propionate, and the like. Of course, these compositions may also contain minor amounts of nontoxic, auxiliary substances, such as wetting or emulsifying agents, pH buffering agents, and so forth.

Methods for the preparation of compositions comprising the compounds of the invention include formulating the derivatives with one or more inert, pharmaceutically acceptable carriers to form either a solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein.

A carrier of the invention can be one or more substances which also serve to act as a diluent, flavoring agent, solubilizer, lubricant, suspending agent, binder, or tablet disintegrating agent. A carrier can also be an encapsulating material.

In powder forms of the invention's compositions, the carrier is preferably a finely divided solid in powder form which is interdispersed as a mixture with a finely divided powder from of one or more compound. In tablet forms of the compositions, one or more compounds is intermixed with a carrier with appropriate binding properties in suitable proportions followed by compaction into the shape and size desired. Powder and tablet form compositions preferably contain between about 5 to about 70% by weight of one or more compound. Carriers that may be used in the practice of the invention include, but are not limited to, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The compounds of the invention may also be encapsulated or microencapsulated by an encapsulating material, which may thus serve as a carrier, to provide a capsule in which the derivatives, with or without other carriers, is surrounded by the encapsulating material. In an analogous manner, cachets comprising one or more compounds are also provided by the instant invention. Tablet, powder, capsule, and cachet forms of the invention can be formulated as single or unit dosage forms suitable for administration, optionally conducted orally.

If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

Formulations for parenteral administration may be in the form of aqueous or non-aqueous sterile injection solutions or suspensions. These solutions and suspensions can be prepared from sterile powders or granules having one or more of the carriers or diluents mentioned for use in the formulations for oral administration. The compounds can be dissolved or suitably emulsified in water, polyethylene glycol, propylene glycol, ethanol, corn oil, cottonseed oil, peanut oil, sesame oil, benzyl alcohol, sodium chloride, and/or various buffers. Other adjuvants and modes of administration are widely known in the pharmaceutical art.

For oral administration, the pharmaceutical composition can be in the form of, for example, a tablet, capsule, a soft gelatin (softgel) capsule, a hard gelatin capsule, suspension or liquid. The pharmaceutical composition is preferably made in the form of a dosage unit containing a particular amount of the active ingredient. Examples of such dosage units are tablets or softgel capsules. The active ingredient can also be administered by injection as a composition wherein, for example, saline, dextrose or water can be used as a suitable carrier.

Soft gelatin capsules can be prepared in which capsules contain a mixture of the agonist, the opioid antagonist-polymer conjugate, and oleaginous and/or non-aqueous, and/or water miscible solvents such as polyethylene glycol and the like. Hydrophilic solvents compatible with softgel capsules can include PEG400, PEG800, ethanol, glycerin, PPG, polysorbates, povidone (PVP), and the like containing up to about 5-8% water. The softgel capsules can optionally contain a buffer, a co-solvent, or a nucleophile. Hard gelatin capsules can contain mixtures of the agonist, the polymer-antagonist conjugate in combination with a solid, pulverulent carrier, such as, for example, lactose, saccharose, sorbitol, mannitol, potato starch, corn starch, amylopectin, cellulose derivatives, or gelatin.

In suppository forms of the compositions, a low-melting wax such as, but not limited to, a mixture of fatty acid glycerides, optionally in combination with cocoa butter is first melted. One or more compounds are then dispersed into the melted material by, as a non-limiting example, stirring. The non-solid mixture is then placed into molds as desired and allowed to cool and solidify.

Non-limiting compositions in liquid form include solutions suitable for oral or parenteral administration, as well as suspensions and emulsions suitable for oral administration. Sterile aqueous based solutions of one or more compounds, optionally in the presence of an agent to increase solubility of the derivative(s), are also provided. Non-limiting examples of sterile solutions include those comprising water, ethanol, and/or propylene glycol in forms suitable for parenteral administration. A sterile solution of the invention may be prepared by dissolving one or more compounds in a desired solvent followed by sterilization, such as by filtration through a sterilizing membrane filter as a non-limiting example. In another embodiment, one or more compounds are dissolved into a previously sterilized solvent under sterile conditions.

A water based solution suitable for oral administration can be prepared by dissolving one or more compounds in water and adding suitable flavoring agents, coloring agents, stabilizers, and thickening agents as desired. Water based suspensions for oral use can be made by dispersing one or more compounds in water together with a viscous material such as, but not limited to, natural or synthetic gums, resins, methyl cellulose, sodium carboxymethyl cellulose, and other suspending agents known to the pharmaceutical field.

Pulmonary administration can be achieved by inhalation or by the introduction of a delivery device into the pulmonary system, e.g., by introducing a delivery device which can dispense (wet or dry) the pharmaceutical composition. The BMPR2 activator or its combination with at least one other active compound can be provided in a dispenser which delivers the composition in a form sufficiently small such that it can be inhaled.

In therapeutic use, the compounds of the invention are administered to a subject at dosage levels of from about 0.05 mg/kg to about 8.0 mg/kg of body weight per day. For a human subject of approximately 70 kg, this is a dosage of from 4 mg to 600 mg per day. In another aspect, the compounds of the invention are administered to a subject at dosage levels of from about 0.05 mg/kg to about 100.0 mg/kg of body weight per day. Such dosages, however, may be altered depending on a number of variables, not limited to the activity of the compound used, the condition to be treated, the mode of administration, the requirements of the individual subject, the severity of the condition being treated, and the judgment of the practitioner.

The foregoing ranges are merely suggestive, as the number of variables in regard to an individual treatment regime is large, and considerable excursions from these recommended values are not uncommon.

### V. Methods of Use

A compound of the invention, such as ascomycin, or a pharmaceutically acceptable solvate, salt, analog, or prodrug thereof, can be administered to a subject upon determination of the subject as having pulmonary hypertension, in particular pulmonary arterial hypertention, or unwanted condition that would benefit by treatment with said compound. The determination can be made by medical or clinical personnel as part of a diagnosis of a disease or condition in a subject.

For administration to non-human animals, the drug or a pharmaceutical composition containing the drug may also be added to the animal feed or drinking water. It will be convenient to formulate animal feed and drinking water products with a predetermined dose of the drug so that the animal takes in an appropriate quantity of the drug along with its diet. It will also be convenient to add a premix containing the drug to the feed or drinking water approximately immediately prior to consumption by the animal.

### Kits/Articles of Manufacture

For use in the therapeutic applications described herein, kits and articles of manufacture are also within the scope of the invention. Such kits can comprise a carrier, package, or container that is compartmentalized to receive one or more containers such as vials, tubes, and the like, each of the container(s) comprising one of the separate elements to be used in a method of the invention. Suitable containers include, for example, bottles, vials, syringes, and test tubes. The containers can be formed from a variety of materials such as glass or plastic.

For example, the container(s) can comprise one or more compounds of the invention, optionally in a composition or in combination with another agent as disclosed herein. The container(s) optionally have a sterile access port (for example the container can be an intravenous solution bag or a vial having a stopper pierceable by a hypodermic injection needle). Such kits optionally comprising a compound with an identifying description or label or instructions relating to its use in the methods of the present invention.

A kit of the invention will typically may comprise one or more additional containers, each with one or more of various materials (such as reagents, optionally in concentrated form, and/or devices) desirable from a commercial and user standpoint for use of a compound of the invention. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

The terms “kit” and “article of manufacture” may be used as synonyms.

Having now generally described the invention, the same will be more readily understood through reference to the following examples which are provided by way of illustration, and are not intended to be limiting of the present invention, unless specified.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Example 1

### Pre-Clinical Study

The purpose of this study was to establish pulmonary arterial hypertension (PAH) in rats by induction with Sugen 5416, a vascular endothelial growth factor (VEGF) inhibitor, plus chronic hypoxia (SuHx) in order to evaluate and compare the efficacy of tacrolimus and ascomycin for the treatment of PAH in the SuHx rat model. The assay is designed to measure the pulmonary and systemic arterial blood pressures related to PAH in the anaesthetized SuHx rat induced with a single subcutaneous dose of Sugen 5416, combined with an induction period of chronic hypoxia.

Male Sprague Dawley rats (Charles River Laboratories) weighing between 200 and 250 g at the time of their enrollment in the study were the test subjects for this study. The animals were assigned to groups by the Study Director in order to evenly distribute the ranges of body weights throughout all treatment groups. Animals in the test groups (n=10 in each group) received one subcutaneous injection of 20 mg/kg of SU5416 on Day 0 and were returned to their respective cages. These rats were placed in cages in which the controlled airflow (ventilated cage system) was adjusted to deliver a FiO₂ equivalent to 0.10 (10%) using a mixture of nitrogen and ambient air, for a period of 3 weeks. Animals in the control group (n=5) remained in cages exposed to ambient oxygen levels.

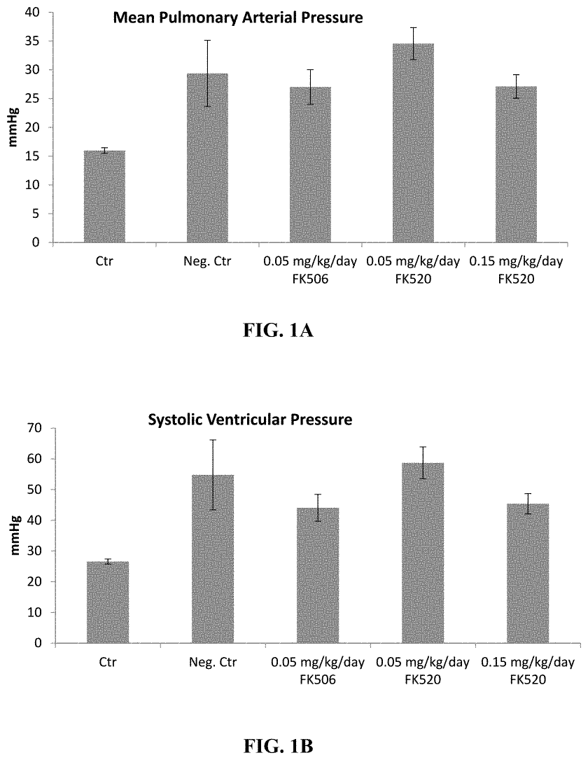

On day 21, animals in the test groups were implanted with osmotic pumps filled with the vehicle alone (negative control), or with the appropriate concentration of tacrolimus (FK506) or ascomycin (FK520) to deliver 0.05 mg/kg/day of tacrolimus, 0.05 mg/kg/day of ascomycin, or 0.15 mg/kg/day of ascomycin for a period of three weeks. At the end of the study, the rats were anaesthetized and hemodynamic data was obtained. The data are shown in FIGS. 1A and 1B. Vehicle-treated rats showed severe PAH, but ascomycin at 0.15 mg/kg/day reversed the elevation in pulmonary arterial pressure (FIG. 1A), and RVSP (FIG. 1B), resulting in decreasing systolic pulmonary arterial pressure and right ventricular systolic pressure. Cardiac output was lower after administration of Sugen5416 and hyposia, and increased with ascomycin treatment.

### Example 2

### Pre-Clinical Study

A second study was conducted as described in Example 1, except the rats injected with Sugen 5416 and placed in hypoxia for 3 weeks were returned to normoxia for three weeks, and then treated for three weeks. The rats were then implanted with s.c. osmotic pumps delivering vehicle (negative control), 0.05 mg/kg/day tacrolimus (FK506), or 0.15 mg/kg/day, 1.0 mg/kg/day, 3.0 mg/kg/day ascomycin (FK520). Rats that were not treated with Sugen 5416 and remained under normoxia served as control. There were 5 rats in the control group, and 10 rats in each treated group.

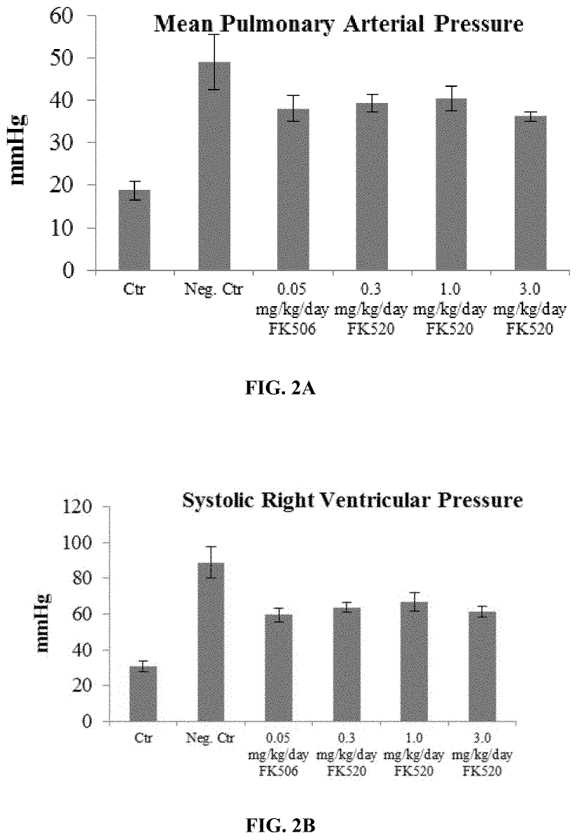

At the end of the study, the rats were anaesthetized and hemodynamic data was obtained. The data is shown in FIGS. 2A and 2B. Hemodynamic data showed that 0.05 mg/kg/day tacrolimus reduces the elevation in right ventricular systolic pressure and right ventricular hypertrophy. Similarly, all three doses of ascomycin showed decreases in systolic pulmonary arterial pressure and right ventricular systolic pressure.

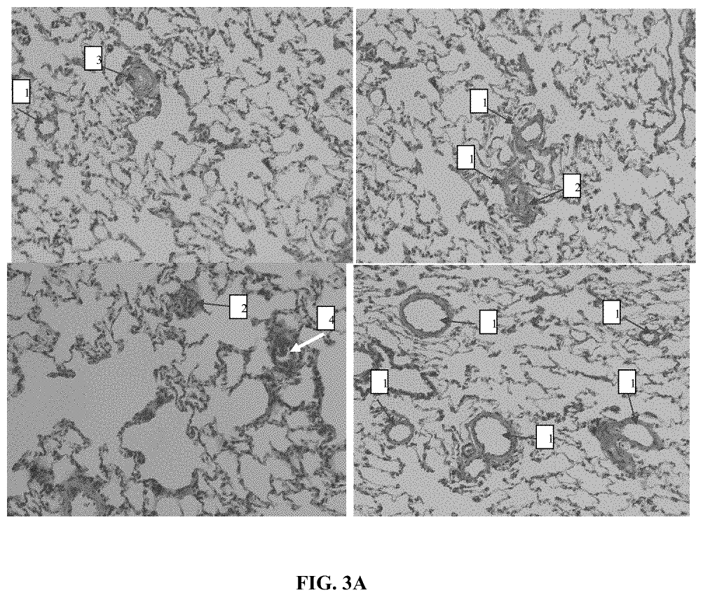

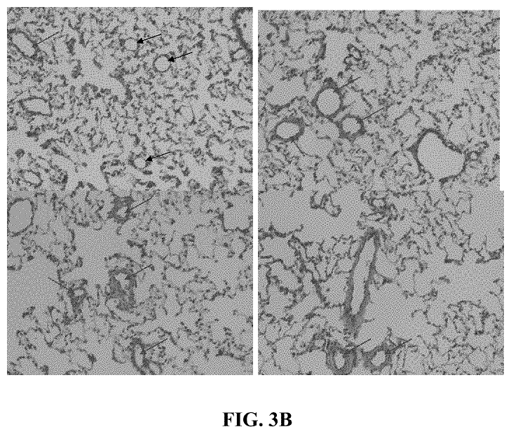

The left lobes of the lungs were harvested from every rat, fixed with 10% formalin, sliced at 5 μm thickness, mounted and stained with Movat staining. The micrographs from control group show that the vessels in the lungs are normal and not muscularized, while the micrographs from the negative control animals (FIG. 3A) show all four grades of disease progression (1—medial hypertrophy, 2—cellular intimal reaction/proliferation, 3—medial hypertrophy and occasional concentric laminar neointimal lesion, and 4—occasional plexiform lesions). Of particular significance in SuHx model is the rare plexiform lesions (label 4 in FIG. 3A) that are neointimal hyperplasia blocking the small vessels resulting in increased pressure. With plexiform lesions and endothelial cells overgrowth, obliteration is formed. On the other hand, the micrographs from 1 mg/kg/day ascomycin treated animals (FIG. 3B) showed mostly medial hypertrophy (label 1) and non-muscular vessels.

Thus, hemodynamic data and histopathology show that 0.3 or 1.0 mg/kg/day ascomycin and 0.05 mg/kg/day tacrolimus have similar efficacies in the SuHx rat model.

### Example 3

### Clinical Study

Patients are invited to participate in this study because they have pulmonary hypertension (PH) and are currently treated with one or multiple drugs for PH such as PDE-5 inhibitors (sildenafil, tadalafil), prostacyclins (Flolan, Remodulin, Iloprost) and/or the endothelin antagonist Ambrisentan. This study is open to male or female subjects, 18-70 years of age, with pulmonary hypertension. The Inclusion and Exclusion criteria used are below:

### Inclusion Criteria

1. Age ≥18 and <70 years
2. Diagnosis of WHO Group I Pulmonary Arterial Hypertension (PAH) (Idiopathic (I)PAH, Heritable PAH (including Hereditary Hemorrhagic Telangiectasia), Associated (A)PAH (including collagen vascular disorders, drugs+toxins exposure, congenital heart disease, and portopulmonary disease).
3. Previous Right Heart Catheterization (RHC) that documented:
   a. Mean PAP ≥25 mmHg.
   b. Pulmonary capillary wedge pressure <15 mmHg.
   c. Pulmonary Vascular Resistance ≥3.0 Wood units or 240 dynes/sec/cm5
5. All NYHA/WHO functional classes.
6. Willingness of female subjects to use birth control, or be post-menopausal, or status post hysterectomy.

### Exclusion Criteria:

1. WHO Group II-V Pulmonary Hypertension.
2. Current or prior experimental PAH treatments within the last 6 months (including but not limited to tyrosine kinase inhibitors, rho-kinase inhibitors, or cGMP modulators).
3. Total lung Capacity (TLC)<60% predicted; if TLC b/w 60 and 70% predicted, high resolution computed tomography must be available to exclude significant interstitial lung disease.
5. Forced expiratory volume (FEV1)/Forced Vital Capacity (FVC)<70% predicted and FEV1<60% predicted
6. Significant left-sided heart disease (based on screening Echocardiogram):
   a. Significant aortic or mitral valve disease
   b. Diastolic dysfunction ≥Grade II
   c. Left Ventricle (LV) systolic function <45%
   d. Pericardial constriction
   e. Restrictive cardiomyopathy
   f. Significant coronary disease with demonstrable ischemia.
7. Chronic renal insufficiency defined as an estimated creatinine clearance <30 ml/min (by Modification of diet in renal disease (MDRD) equation).
8. Current atrial arrhythmias not under optimal control.
9. Uncontrolled systemic hypertension: SBP>160 mm or DBP>100 mm
10. Severe hypotension: SBP<80 mmHg.
11. Pregnant or breast-feeding.
12. Psychiatric, addictive, or other disorder that compromises patient's ability to provide informed consent, to follow study protocol, and adhere to treatment instructions.
13. Active cyclosporine use.
14. Known allergy or hypersensitivity to ascomycin.
15. Planned initiation of cardiac or pulmonary rehabilitation during period of study.
16. Human Immunodeficiency Virus infection.
17. Moderate to severe hepatic dysfunction with a Child Pugh score >10.
18. Hyperkalemia defined as Potassium >5.1 mEq/L at screening.
19. Known active infection requiring antibiotic, antifungal, or antiviral therapies.
20. Co-morbid conditions that would impair a patient's exercise performance and ability to assess WHO functional class, including but not limited to chronic low-back pain or peripheral musculoskeletal problems.

Participation in the study lasts for approximately 16 weeks. During this time, patients will be required to visit the clinic approximately 4-5 times. The blood will be drawn shipped to a testing lab to measure ascomycin levels. The goal is to achieve trough blood levels of 5-15 ng/mL of ascomycin. Patients will receive the study drug for the duration of study. The drug will be delivered in a prepared bottle, which allows monitoring of drug intake. This device is called a Medication Event Monitoring System (MEMS) and for it to monitor drug intake properly. Patients will be instructed to take out one tablet at a time from the bottle.

If a patient agrees to take part in this study, they will first sign this consent form. After the patients have signed, dated and received a copy of this consent form, they will have the study screening visit to ensure the patient is eligible to take part in this study. Previous test results (echocardiogram, physical examination, pulmonary function tests, Right Heart Catheterization (RHC) may also be used to determine patient eligibility.

The primary efficacy endpoint is change from baseline in 6 minute walk distance (6MWD) or change in pulmonary vascular resistance (PVR) evaluated after treatment compared to placebo.

The secondary efficacy endpoints includes:

(a) time to clinical worsening of PAH, as defined by the time from randomization to the first occurrence of death, lung transplantation, hospitalization for PAH, atrial septostomy, study discontinuation due to addition of other PAH therapeutic agents, or study discontinuation due to 2 or more early escape criteria;

(b) change from baseline measured after treatment compared to placebo in:
(i) WHO functional class;
(ii) SF-36® health survey physical functioning scale;
(iii) BDI immediately following exercise; and/or
(iv) an assessment of the safety and tolerability of the study drug; and

(c) change from baseline measured after treatment compared to placebo in plasma levels of NT-ProBNP, BMP and cTnT.

### Results

The results show that ascomycin is well tolerated at all tested doses, there are no drug-related serious adverse events, there are no incidences of hypertension or cardiovascular events, and biomarker data indicated an increase in BMPR2 signaling.

The data show that treatment of patients with ascomycin improves the pulmonary function of the patients as measured by an increase in the 6MWD, decrease in PVR, and adjusts the molecular biomarkers that can be predictive of clinical outcome towards a more normal level as measured by a very significant decrease in the NT-proBNP levels. Thus, the use of an agent that increases the BMPR2 signaling is efficacious for treating PAH.

While the invention has been particularly shown and described with reference to a preferred embodiment and various alternate embodiments, it will be understood by persons skilled in the relevant art that various changes in form and details can be made therein without departing from the spirit and scope of the invention. All printed patents and publications referred to in this application are hereby incorporated herein in their entirety by this reference.

We claim:

1. A method of treating or preventing pulmonary hypertension in a patient in need thereof, the method comprising:
administering a therapeutically effective amount of a compound that increases BMPR2 signaling (BMPR2 activator) to the patient with pulmonary hypertension, wherein the compound is an ascomycin, or a pharmaceutically acceptable salt, solvate, analog, or prodrug thereof.

2. The method of claim 1, wherein the compound is administered to improve exercise ability, delay clinical worsening, or combinations thereof.

3. The method of claim 1, wherein the pulmonary hypertension is associated with one or more of chronic obstructive pulmonary disease (COPD), sleep-disordered breathing, an alveolar hypoventilation disorder, chronic exposure to high altitude, a developmental abnormality, thromboembolic obstruction of proximal and/or distal pulmonary arteries, a non-thrombotic pulmonary embolism, sarcoidosis, histiocytosis X, lymphangiomatosis or compression of pulmonary vessels.

4. The method of claim 1, wherein the pulmonary hypertension is pulmonary arterial hypertension (PAH).

5. The method of claim 1, wherein the patient experiences at least one of (a) adjustment of one or more hemodynamic parameters indicative of improvement of the pulmonary hypertension condition towards a more normal level versus baseline; (b) increase in exercise capacity versus baseline; (c) lowering of Borg dyspnea index (BDI) versus baseline; (d) improvement of one or more quality of life parameters versus baseline; (e) movement to a lower WHO functional class; and (f) a reduction in plasma natriuretic peptide levels versus baseline.

6. The method of claim 1, wherein the daily dose provides serum concentration of about 0.02 ng/mL to about 50 ng/mL.

7. The method of claim 1, wherein administration is oral, intra venus, or inhalation.

8. The method of claim 1, wherein the compound is ascomycin, desmethyl ascomycin (FK523), or prolyltacrolimus (FK525).

9. A pharmaceutical composition comprising an ascomycin or a pharmaceutically acceptable salt, solvate, analog, or prodrug thereof, and at least one pharmaceutically acceptable excipient.
